(19) United States
(12) Patent Application Publication
(10) Pub. No.: US 2020/0060871 A1
(43) Pub. Date: Feb. 27, 2020

(54) ICE CLAMP

(71) Applicant: Stephen Albanese, Pompano Beach, FL (US)

(72) Inventor: Stephen Albanese, Pompano Beach, FL (US)

(21) Appl. No.: 16/111,977

(22) Filed: Aug. 24, 2018

(51) Int. Cl. A61F 7/10

(52) U.S. Cl. CPC A61F 7/10; A61F 2007/0043

(57) ABSTRACT

An ice clamp removably secures a cold pack to various areas of a user's body and includes first and second opposing clamp members that are pivotally moveable relative to one another. Each of the clamp members includes a jaw, a handle extending from the jaw, and at least one hinge member for allowing hinged movement of the clamp members relative to one another whereby the jaws are moved between a relaxed, closed position and an open position against the force of a spring. A cold pack is removably attachable to the clamp members, and against inner concave surfaces of the opposing jaws, so that when the ice clamp is secured to a desired area of the user's body, the cold pack is held securely against the desired area to administer ice therapy. In a preferred embodiment, the cold pack includes an outer fabric comfort sleeve.

### BACKGROUND OF THE INVENTION

### Field of the Invention

The present invention relates to a device for holding an ice pack on a user's body/extremities and, more particularly, to a clamp device having opposing jaw members that are urged closed by a spring for holding an ice pack against various areas of a user's body, such as joints and extremities.

### Discussion of the Related Art

Overuse or injury to muscles, tendons and ligaments typically results in inflammation that causes pain. The best way to relieve the pain is to reduce or eliminate the inflammation. This can be achieved by taking over-the-counter nonsteroidal anti-inflammatory drugs (NSAIDs), such as aspirin and ibuprofen. However, there are concerns about regularly ingesting chemical drugs into one's body, as well as the possible side effects caused by drug products, including NSAIDs.

Another commonly used treatment to reduce pain caused by inflammation is to apply cold temperature, such as an ice pack, to the inflamed area. In fact, in most cases, the application of cold temperature to inflamed areas of the body is the best treatment method since it is fast acting, inexpensive and has no significant side effects.

For those who regularly engage in sports and physical fitness activities, or other strenuous work, sore joints and muscles are a common occurrence. The use of ice packs (cold packs), as well as ice baths, can be very effective in treating the recovery of muscles, joints and tissues so that people can quickly return to enjoying their favorite activities.

While application of ice packs (cold packs) to various areas of the human body is highly effective to reduce inflammation and pain, it is often difficult to secure an ice pack on certain areas of the body. In the past, others have proposed devices that hold an ice bag or frozen cold pack in place on, for example, a person's knee, shoulder or elbow. In many cases, these devices include straps that need to be wrapped around the user's body in order to effectively hold the device and ice bag in place. These devices can be extremely awkward to handle, and usually require at least two hands to operate. In many instances, a user is not able to apply these devices without the assistance of another person, as it is difficult to secure the device and ice pack in place, while simultaneously wrapping the straps or other securing devices around the user's body. For example, when placing one of these devices on an elbow, it is physically impossible to use the hand on the same arm as the elbow to assist in application of the device to the user's elbow.

Accordingly, there remains a definite need for a device that effectively secures an ice pack or cold pack to various areas of a human body, and wherein the device is easy to attach and remove to the body by the user. Additionally, compression is another treatment to improve muscle, tissue and joint recovery quicker. This device will add compression while icing.

### Objects and Advantages of the Present Invention

Considering the forgoing, it is a primary object of the present invention to provide a clamp device for securing an ice pack or cold pack to various areas of a user's body, and wherein the device is easy to operate with one hand by the user to attach and remove the clamp device and ice pack/cold pack to the various selected areas of the body.

It is a further object of the present invention to provide a clamp device that effectively secures an ice pack/cold pack to various areas of a user's body, and wherein the clamp device is adapted for easy and convenient removable attachment of an ice pack within the clamp jaws of the device.

It is still a further object of the present invention to provide a clamp device for securing an ice pack/cold pack to various areas of the human body, and wherein the clamp device is relatively simple in construction, easy to operate and relatively easy to manufacture.

It is still a further object of the present invention to provide a clamp device for removably securing an ice pack/cold pack to various areas of the human body including, but not limited to, feet, ankles, calves, knees, thighs, wrists, forearms, elbows, biceps and shoulders.

These and other objects and advantages of the present invention are more readily apparent with reference to the following detailed description taken in conjunction with the accompanying drawings.

### SUMMARY OF THE INVENTION

The present invention is directed to an ice clamp for removably securing a cold pack to various areas of a user's body. The ice clamp is specifically designed to allow attachment and removal of the ice clamp and cold pack to various areas of the user's body with the use of one hand of the user. The ice clamp includes first and second opposing clamp members, that are essentially identical and are arranged in opposing relation to one another. Each of the first and second clamp members includes a jaw, a handle extending from the jaw, and at least one hinge member extending inwardly from the handle for hingedly attaching the first and second clamp members together as a single unit, while allowing hinged movement of the first and second clamp members relative to one another to move the jaws open and closed. Each of the jaws includes a distal end portion, a proximal end portion, an inner concave surface, an outer convex surface, and opposite side edges. The inner concave surfaces of the jaws are positioned in opposing, confronting relation to one another and in surrounding relation to an open area for receiving a portion of the user's body therein. A spring is cooperatively coupled with the hinge member for urging the jaws towards one another to a relaxed state, defining the closed position. The handles of the clamp members each include a pressure application area for applying an inwardly directed squeezing force thereto, thereby causing hinged movement of the first and second clamp members from the closed position to the open position. More particularly, the jaws are moved away from one another to increase the separation between the distal end portions of the jaws, to allow placement and removal of the ice clamp over the various selected areas of the user's body such as, but not limited to, feet, ankles, calves, knees, thighs, wrists, forearms, elbows, biceps and shoulders.

A cold pack is removably attachable to the inner concave surfaces of the opposing jaws so that when the ice clamp is secured on the user, the cold pack is held against the user's body. The cold pack includes a liquid impervious casing having one or more chambers that are filled with a freezable substance. The cold pack further includes an outer fabric comfort sleeve for containing the liquid impervious casing therein. The cold pack is releasably secured to the inner concave surfaces of the jaws with the use of releasable fasteners such as, but not limited to, hook and loop fasteners, snap fasteners, belt fasteners, elastic bands, hooks or other well-known releasable fasteners.

### BRIEF DESCRIPTION OF THE DRAWINGS

For a fuller understanding of the nature of the present invention, reference should be made to the following detailed description taken in conjunction with the accompanying drawings in which:

FIG. 1 is a perspective view showing the ice clamp of the present invention secured to a user's shoulder for holding a cold pack against the user's shoulder and upper arm;

FIG. 2 is a front elevational view of the ice clamp of the present invention;

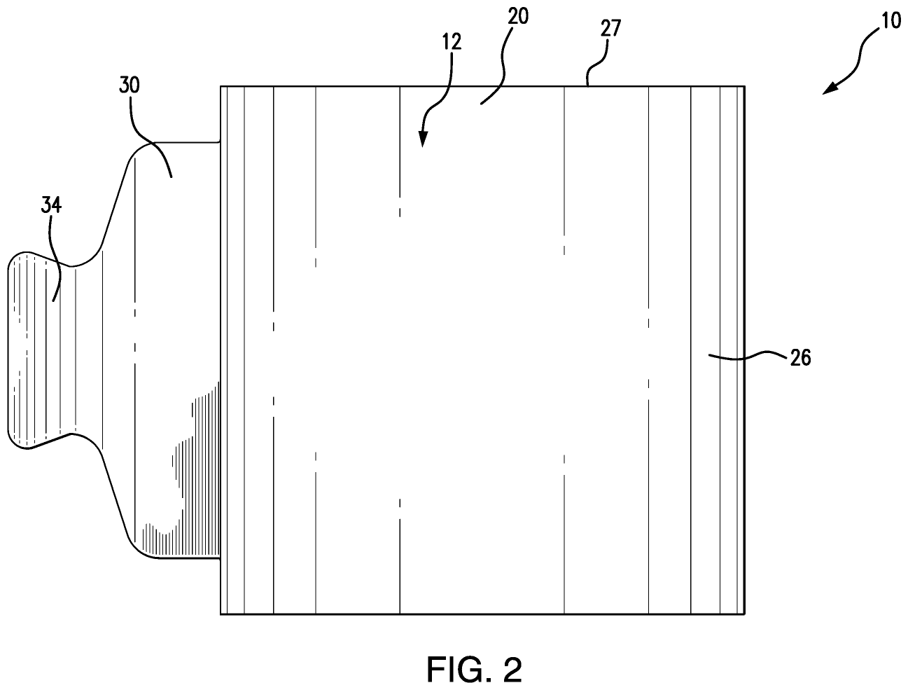

FIG. 3 is a side elevational view of the ice clamp showing a cold pack secured within the jaws of the clamp, with the jaws shown in a closed, relaxed state;

FIG. 4 is a side elevational view showing the jaws of the ice clamp moved to an open position, upon application of squeezing inward pressure on the handles of the ice clamp, as indicated by the arrows, to thereby allow for placement and removal of the ice clamp and cold pack onto various areas of the user's body with the use of just one hand;

FIG. 5 is a bottom plan view of the ice clamp, shown with the cold pack removed, and illustrating hook components of a hook and loop releasable fastener fitted to the inner concave surface of the jaws of the clamp to allow removable attachment of the cold pack to the jaws of the ice clamp;

FIG. 6 is a top plan view of a cold pack shown being inserted within a cold pack fabric sleeve, for subsequent attachment to the inner concave surfaces of the opposing jaws of the ice clamp of the present invention; and

FIG. 7 is a perspective view showing the ice clamp of the present invention fitted to a leg of a user for holding the cold pack against the user's lower leg.

Like reference numerals refer to like parts throughout the several views of the drawings.

### DETAILED DESCRIPTION OF THE PREFERRED EMBODIMENT

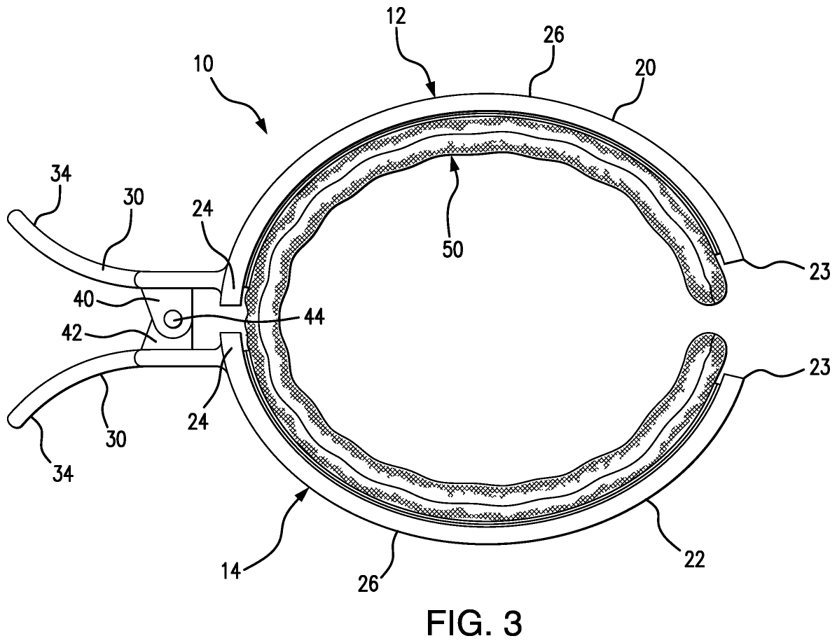

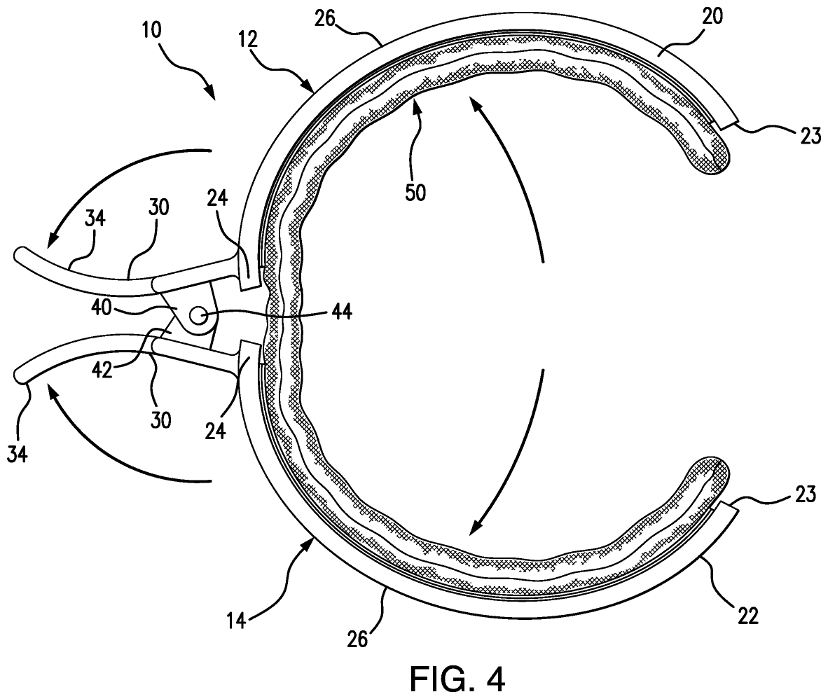

Referring to the several views of the drawings, the ice clamp of the present invention is shown and is generally indicated as 10. The ice clamp 10 includes first and second opposing clamp members 12, 14 that are pivotally connected to one another to allow for pivoting movement of the clamp members 12, 14 relative to one another. The first clamp member includes a jaw 20 and a handle 30. Similarly, the second clamp member 14 includes a jaw 22 and a handle 30. The jaws 20, 22 are arranged in opposing relation to one another, as seen in FIGS. 3 and 4. Each of the jaws 20, 22 includes a proximal end portion 24, a distal end portion 23, an inner concave surface 25, an outer convex surface 26 and opposite side edges 27, 28. The handles 30 extend from the proximal end portions 24 of the jaws 20, 22 and include a pressure application surface 34 for applying an inwardly directed squeezing pressure thereto to cause pivoting movement of the clamp members 12, 14 relative to one another, and particularly opening of the jaws 20, 22.

Hinge members 40, 42 extend from the handles 30 of the first and second opposing clamp members, as seen in FIGS. 3 and 4. The hinge members 40, 42 are pivotally connected by a hinge pin 44 that allows hinged, pivoting movement of the hinge members 40, 42 about the hinge pin 44. A spring 46 is fitted to the hinge pin 44 and engages the first and second opposing clamp members for applying a force that urges the opposing clamp members, and particularly the jaws 20, 22, towards one another to a relaxed state defining a closed position, as seen in FIG. 3. Upon application of the inwardly directed squeezing force to the pressure application area 34 of the handles, as illustrated in FIG. 4, the jaws 20, 22 of the clamp members are moved away from one another, as the first and second clamp members are pivoted about the hinge pin 44 of the hinge structure extending from the handles, causing the distal end portions 23 of the jaws 20, 22 to separate further away from one another. This allows a portion of the user's body, such as an area along the arms or legs, to be inserted through the gap between the distal end portions 23 of the jaws and into the open area between the jaws. Once the ice clamp 10 is properly positioned at the desired area on the user's body, the squeezing pressure is released from the pressure application areas 34 on the handles, whereby the spring 46 urges the jaws 20, 22 towards the closed position until the opposing clamp members 12, 14 reach either the fully closed position or the clamp members (i.e., the cold pack within the jaws of the clamp members) engage the user's body, whereby further closing of the jaws is prevented.

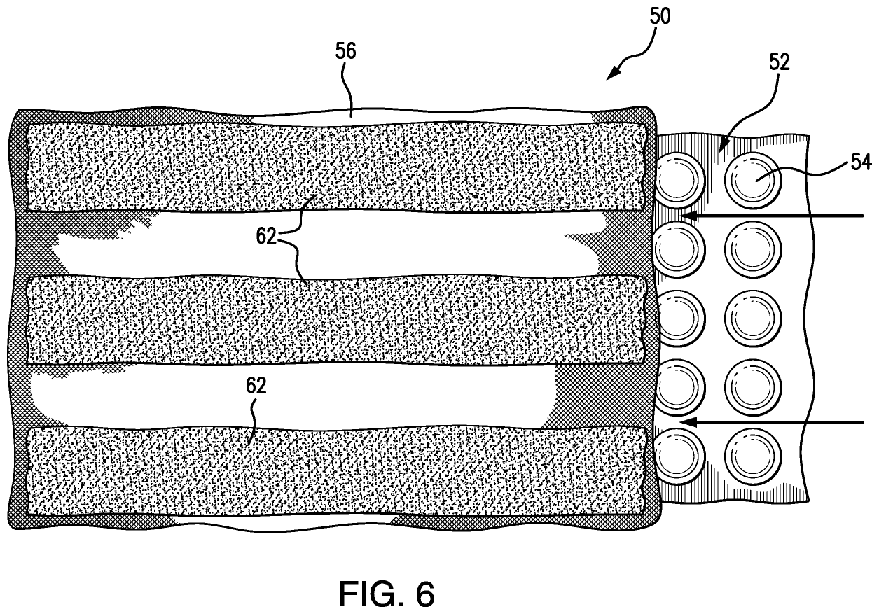

Referring to FIGS. 3, 4 and 6, a cold pack 50 is specifically sized, structured and configured for attachment to the jaws 20, 22, and specifically the inner concave surfaces 25 of the jaws. In the preferred embodiment, the cold pack 50 includes a liquid impervious casing 52 that includes one or more chambers 54 that are filled with a freezable substance. The casing 52 is inserted within a comfort sleeve 56 formed of a soft fabric that is particularly suited for engagement against the user's skin, but yet allows for the cold temperature of the freezable substance 54 to pass through the sleeve 56 to apply cold (ice) therapy to the user.

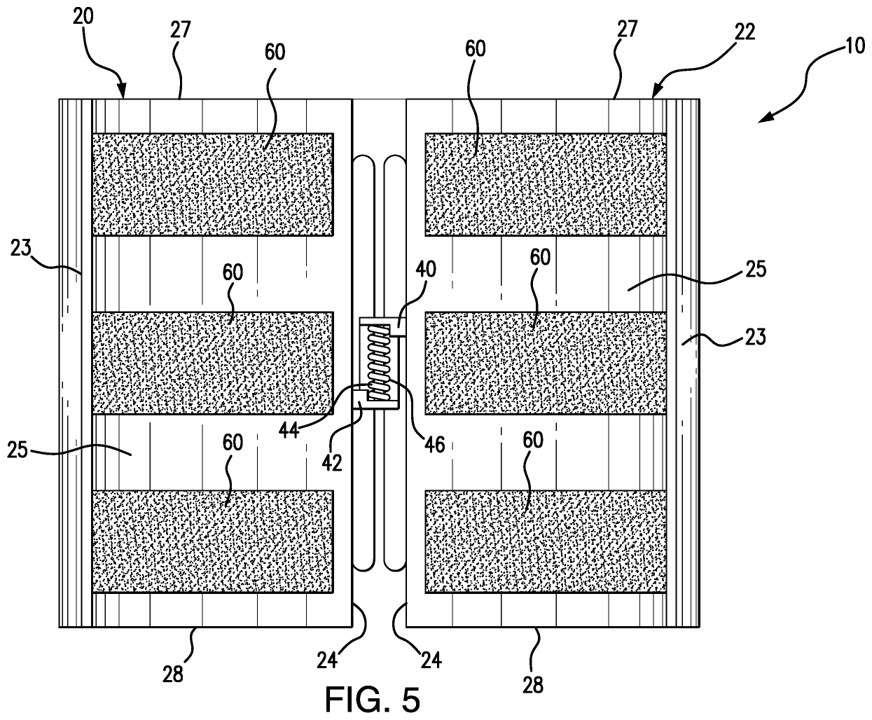

The cold pack 50 is attachable to the first and second clamp members, and particularly the inner concave surfaces 25 of the jaws 20, 22 with the use of releasable fasteners. While any of a number of types of releasable fasteners are contemplated within the spirit and scope of the invention, the embodiment shown in the drawings provides for a hook and loop fastener system. Specifically, hook components 60 of a hook and loop releasable fastener are bonded to the inner concave surfaces 25 of the opposing jaws 20, 22, as seen in FIG. 5. These hook components 60 may be sufficient to releasably attach to the fabric of the sleeve 56 of the cold pack 50. Alternatively, the sleeve 56 of the cold pack may be provided with corresponding loop components 62 for releasable attachment to the correspondingly positioned hook components 60. The releasable fasteners allow for quick and easy removal and replacement of cold packs to the jaws 20, 22 of the ice clamp 10. Accordingly, after each use, the cold pack 50 can be quickly removed from the clamp members 12, 14 and placed in a freezer for subsequent use. When needed, the cold pack can then be removed from the freezer and quickly attached to the jaws 20, 22, in the manner shown in FIGS. 3 and 4 so that the ice clamp 10 can be fitted to the desired area of the user's body to hold the cold pack 50 against the desired body area for the appropriate ice therapy time, which is typically between 10-20 minutes.

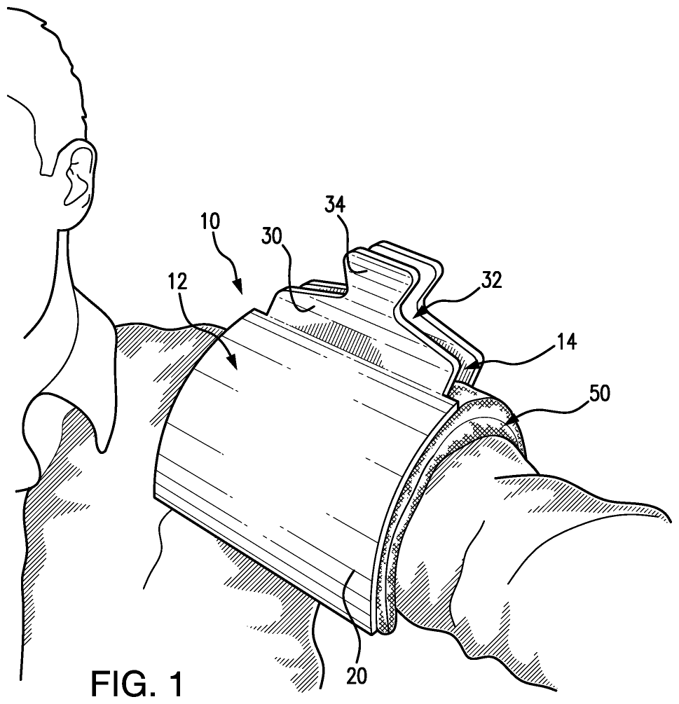

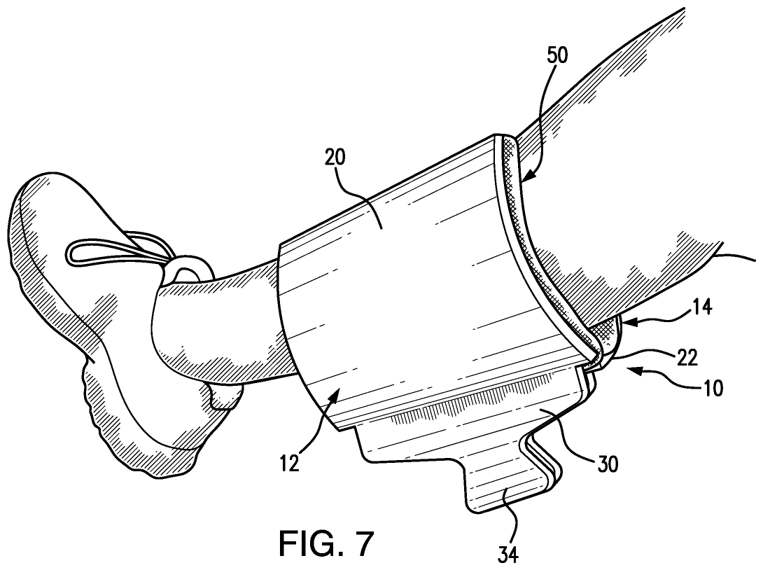

FIG. 1 shows the ice clamp 10 holding the cold pack 50 against the user's shoulder and upper arm area. FIG. 7 illustrates another example of an attached position of the ice clamp, wherein the ice clamp 10 is secured to a user's calf, below the knee, for holding the ice pack 50 at the desired position to administer ice therapy.

While the present invention has been shown and described in accordance with a preferred and practical embodiment, it is recognized that departures from the instant disclosure are fully contemplated within the spirit and scope of the present invention which is not to be limited except as defined in the following claims as interpreted under the Doctrine of Equivalents.

What is claimed is:

1. An ice clamp comprising:
first and second opposing clamp members, each clamp member including a jaw, a handle extending from the jaw, and at least one hinge member for hingedly attaching the first and second clamp members together as a single unit;
each of the jaws including a distal end portion, a proximal end portion, an inner concave surface, an outer convex surface, and opposite side edges, and the inner concave surfaces of the jaws positioned in opposing relation to one another and surrounding an open area for receiving a portion of the user's body therein;
a spring for urging the jaws towards one another to a relaxed state defining a closed position;
the handles of the clamp members including a pressure application area for applying an inwardly directed squeezing force thereto to cause hinged movement of the first and second clamp members that moves the jaws away from one another and an increased separation of the distal end portions from one another, against a biased force of the spring, to define an open position for receiving the portion of the user's body between the jaws;
a cold pack including a liquid impervious casing filled with a freezable substance and an outer fabric sleeve for containing the liquid impervious casing therein; and
releasable fasteners on at least the clamp members for removably attaching the cold pack to the inner concave opposing surfaces of the jaws.

2. The ice clamp as recited in claim 1 wherein the at least one hinge member on each of the first and second opposing clamp members extends from the handle.

3. The ice clamp as recited in claim 2 wherein the at least one hinge member on the first clamp member and the at least on hinge member on the second clamp member are pivotally connected to one another by a pivot pin.

4. The ice clamp as recited in claim 3 wherein the spring is fitted over the pivot pin and engages the first and second clamp members for exerting the spring biased force on the first and second clamp members, urging the first and second clamp members to the closed position.

5. The ice clamp as recited in claim 4 wherein the handles on each of the first and second clamp members extend from the proximal end portion of the jaw.

6. The ice clamp as recited in claim 1 wherein the releasable fasteners comprise at least one hook component of a hook and loop fastener, and the at least one hook component being bonded to the inner concave surface of each of the jaws.

7. The ice clamp as recited in claim 6 wherein the outer fabric sleeve of the cold pack includes at least one loop component of a hook and loop fastener for releasable attachment to the at least one hook component on the inner concave surface of each of the jaws.

8. An ice clamp comprising:
first and second opposing clamp members, each clamp member including a jaw, a handle extending from the jaw, and at least one hinge member extending from the handle for hingedly attaching the first and second clamp members together as a single unit;
each of the jaws including a distal end portion, a proximal end portion, an inner concave surface, an outer convex surface, and opposite side edges, and the inner concave surfaces of the jaws positioned in opposing relation to one another and surrounding an open area for receiving a portion of the user's body therein;
a spring engaging the clamp members for urging the jaws towards one another to a relaxed state defining a closed position;
the handles of the clamp members including a pressure application area for applying an inwardly directed squeezing force thereto to cause hinged movement of the first and second clamp members that moves the jaws away from one another and an increased separation of the distal end portions from one another, against a biased force of the spring, to define an open position for receiving the portion of the user's body between the jaws;
a cold pack including a liquid impervious casing filled with a freezable substance and an outer fabric sleeve for containing the liquid impervious casing therein; and
releasable fasteners on at least the clamp members for removably attaching the cold pack to the inner concave opposing surfaces of the jaws.

9. The ice clamp as recited in claim 8 wherein the at least one hinge member on the first clamp member and the at least one hinge member on the second clamp member are pivotally connected to one another by a pivot pin.

10. The ice clamp as recited in claim 9 wherein the spring is fitted over the pivot pin and engages the first and second clamp members for exerting the spring biased force on the first and second clamp members, urging the first and second clamp members to the closed position.

11. The ice clamp as recited in claim 10 wherein the handles on each of the first and second clamp members extend from the proximal end portion of the jaw.

12. The ice clamp as recited in claim 8 wherein the releasable fasteners comprise at least one hook component of a hook and loop fastener, and the at least one hook component being bonded to the inner concave surface of each of the jaws.

13. The ice clamp as recited in claim 12 wherein the outer fabric sleeve of the cold pack includes at least one loop component of a hook and loop fastener for releasable attachment to the at least one hook component on the inner concave surface of each of the jaws.

14. The ice clamp as recited in claim 8 wherein the cold pack surrounds the open area between the jaws when attached to the inner concave opposing surfaces of the jaws.
